(19) United States
(12) Patent Application Publication
(10) Pub. No.: US 2012/0308743 A1
(43) Pub. Date: Dec. 6, 2012

(54) Sefe Visor

(76) Inventor: Seferino Rivera, III, Sugar Land, TX (US)

(21) Appl. No.: 13/454,030

(22) Filed: Apr. 23, 2012

Related U.S. Application Data
(60) Provisional application No. 61/517,589, filed on Apr. 22, 2011.

(51) Int. Cl. B44F 1/10; A61F 9/02; B62J 17/04; A42B 3/22

(52) U.S. Cl. 428/29; 2/424; 2/431; 296/78.1

(57) ABSTRACT

The invention provides dimensional moving photo holographic images in a clear or tinted, polarizing polymeric film for applying to sports equipment such as motorcycle helmets, windshield visors, snow goggles, windshields for motorcycles, mopeds, ATVs, dirt bikes, snowmobiles and watercraft, for example.

### Claim of Priority

This patent application claims priority under 35 USC 119 (e) (1) from U.S. Provisional Patent Application Ser. No. 61/517,589 filed Apr. 22, 2011, of common inventorship herewith entitled, “Sefe Visor.”

### FIELD OF THE INVENTION

The present invention pertains to the field of polyester film products containing images, and more specifically to the field of moving three dimensional holographic images in a clear or tinted, polarizing polymeric thin film applied to cycling helmets, visors, ski goggles, windshields, etc.

### BACKGROUND OF THE INVENTION

The prior art has put forth several designs for cyclist helmets, and tint and image applications. Among these are:

U.S. Pat. No. 5,269,858 to Gary S. Silverman describes a method of simulating stained glass art by applying liquid paints to the object which may be a glass window or sheet. The leading paint dries in approximately two to three hours and then colored paints are applied as a covering over the areas which are peripherally defined by the leading paint.

U.S. Pat. No. 5,896,587 to Debra Gentry describes a bicycle helmet having a transparent eye shade and various interchangeable sun shield portions, along with affixed and built in sun shield portions. Stickers of various styles can be adhered to all eye shade portions.

U.S. Pat. No. 5,035,474 to Gaylord E. Moss, Brian D. Cohn, Mao-Jin J. Chern, Lacy G. Cook, and John J. Ferrer describes a binocular holographic helmet mounted display used by pilots while flying in low light level environments. This mounted display also combines infrared or other image detection and instrumentation symbology which enhance a pilot's flight vision.

None of these prior art references describe the present invention.

### SUMMARY OF THE INVENTION

It is an object of the present invention to provide three dimensional moving photo holographic images in a clear or tinted, polarizing polymeric film for applying to sports equipment such as motorcycle helmets, windshield visors, snow goggles, windshields for motorcycles, mopeds, ATVs, dirt bikes, snowmobiles and watercraft, for example.

### BRIEF DESCRIPTION OF THE DRAWINGS

FIG. 1 is a front perspective view showing a standard motorcycle windshield.

FIG. 2 is a front perspective view showing a holographic shield design which blends with the bike's design.

FIG. 3 is a front perspective view showing a holographic shield design of a skull which is distinct from the bike's design.

FIG. 4 is a front perspective view showing a helmet visor shield design which blends with the helmet.

FIG. 5 is a front perspective view showing a holographic shield design of a skull, which is distinct from the helmet design, on an interchangeable helmet shield.

FIG. 6 is a front perspective view showing ski goggles with interchangeable lens containing different holographic designs.

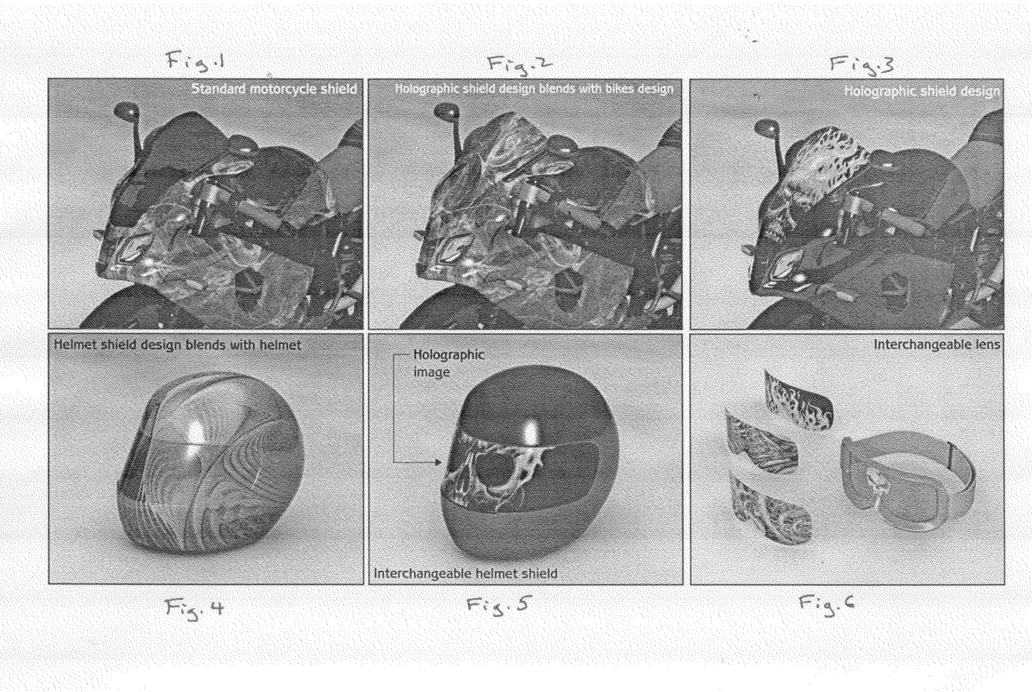

### DETAILED DESCRIPTION OF THE INVENTION

The sky is the limit for today's sport enthusiasts and athletes, and the more extreme the activity, the better. Not only do these sport enthusiasts and athletes enjoy pushing the envelope on the risks they take, but they insist on blazing a trail with a premium of individual style. Skiers and snowboarders may be bundled from head to toe, but still can be distinguished by their gear and by the graphics on their skis and snowboards. Long before the days of chopper builders on reality TV, motorcyclists always have taken pride in individually customizing their bikes, watercraft or other equipment as a personal statement. One thing that skiers, snowboarders, boaters, jet skiers and motorcyclists share is the need for clear vision and vision protection. For skiers and snowboarders the protection takes the form of goggles. For motorcyclists and boaters the protection takes the form of helmet visors and windscreens.

The present invention, hereinafter referred to as the Sefe Visor, offers motorcyclists, snowboarders, boaters and skiers both vision protection and an exciting new venue for expressing individual style. The Sefe Visor is a product line of motorcycle and watercraft windscreens, helmet visors, and snowboard and ski goggles in which the optical quality polymeric matrix of the screen, goggle lens or visor bears a thin polarized film containing an embedded three dimensional moving photo holographic image.

The Sefe Visor incorporates transition lens tints to adjust to strength of light or sun brightness. The image appears more vibrantly as the lens or shield darkens in bright light. The image diminishes and the shield clears with cloudiness or at night time. This image is visible to an onlooker, but does not impede or restrict the outward oriented vision of the cyclist, skier, boater or snowboarder. The Sefe Visor product embeds a moving photo holographic image in a clear or tinted, polarizing polymeric film into the helmet, visor, windshield screen for motorcycles or watercraft or goggles. The present invention combines a high quality motorcycle or watercraft windscreen, a motorcycle helmet visor or a pair of ski and snowboard goggles with the available thin film holographic technology. The consumer can apply graphic arts to their equipment. The Sefe Visor provides suggested artistic design themes from death's heads and gothic dragons to butterflies, eerie clown faces, and anime waifs. The Sefe Visor products are striking and affordable, a viable replacement for an expensive custom airbrush paint job on one's bike, skis, watercraft or snowboard. One embodiment is for the polarizing holographic polymeric films to already be affixed to the windscreens, helmet shields, or goggles, and the consumer purchases the piece of equipment with their preferred graphics. Another embodiment is for the films to be equipped with a peel and stick backing much like a tint for tinting the windows of an automobile and offered as single sheets for the consumer to apply.

The Hungarian-British physicist Dennis Gabor was awarded the Nobel Prize in Physics in 1971 “for his invention and development of the holographic method”. The development of the laser enabled the first practical optical holograms that recorded 3D objects to be made in 1962 by Yuri Denisyuk in the Soviet Union and by Emmett Leith and Juris Upatnieks at University of Michigan, USA. Early holograms used silver halide photographic emulsions as the recording medium. They were not very efficient as the grating produced absorbed much of the incident light. Various methods of converting the variation in transmission to a variation in refractive index (known as “bleaching”) were developed which enabled much more efficient holograms to be produced.

Several types of holograms can be made. Transmission holograms, such as those produced by Leith and Upatnieks, are viewed by shining laser light through them and looking at the reconstructed image from the side of the hologram opposite the source. A later refinement, the “rainbow transmission” hologram, allows more convenient illumination by white light rather than by lasers. Rainbow holograms are commonly seen today on credit cards as a security feature and on product packaging.

Another kind of common hologram, the reflection or Denisyuk hologram, can also be viewed using a white-light illumination source on the same side of the hologram as the viewer and is the type of hologram normally seen in holographic displays. They are also capable of multicolour-image reproduction.

Specular holography is a related technique for making three-dimensional images by controlling the motion of specularities on a two-dimensional surface. It works by reflectively or refractively manipulating bundles of light rays, whereas Gabor-style holography works by diffractively reconstructing wavefronts.

In its early days, holography required high-power expensive lasers, but nowadays, mass-produced low-cost semi-conductor or LED lasers, such as those found in millions of DVD recorders and used in other common applications, can be used to make holograms and have made holography much more accessible to low-budget researchers, artists and dedicated hobbyists.

To make a hologram, the following are required: a suitable object or set of objects, a suitable laser beam, part of the laser beam to be directed so that it illuminates the object (the object beam) and another part so that it illuminates the recording medium directly (the reference beam), enabling the reference beam and the light which is scattered from the object onto the recording medium to form an intereference pattern, a recording medium which converts this interference pattern into an optical element which modifies either the amplitude or the phase of an incident light beam according to the intensity of the interference pattern, an environment which provides sufficient mechanical and thermal stability that the interference pattern is stable during the time in which the interference pattern is recorded.

An existing hologram can be replicated, either optically, similar to holographic recording or in the case of surface relief holograms, by embossing. Surface relief holograms are recorded in photoresists or photothermoplastics and allow cheap mass reproduction. Such embossed holograms are now widely used, for instance, as security features on credit cards or quality merchandise. The Royal Canadian Mint even produces holographic gold and silver coinage through a complex stamping process. The first book to feature a hologram on the front cover was The Skook (Warner Books, 1984) by J P Miller, featuring an illustration by Miller. That same year, “Telstar” by Ad Infinitum became the first record with a hologram cover and National Geographic published the first magazine with a hologram cover.

The first step in the embossing process is to make a stamper by electrodeposition of nickel on the relief image recorded on the photoresist or photothermoplastic. When the nickel layer is thick enough, it is separated from the master hologram and mounted on a metal backing plate. The material used to make embossed copies consists of a polyester base film, a resin separation layer and a thermoplastic film constituting the holographic layer.

The embossing process can be carried out with a simple heated press. The bottom layer of the duplicating film (the thermoplastic layer) is heated above its softening point and pressed against the stamper, so that it takes up its shape. This shape is retained when the film is cooled and removed from the press. In order to permit the viewing of embossed holograms in reflection, an additional reflecting layer of aluminum is usually added on the hologram recording layer. Embossed holograms are used widely on credit cards, banknotes, and high value products.

A holographic image can be obtained using white light in specific circumstances, e.g. with volume holograms and rainbow holograms. The white light source used to view these holograms should always approximate to a point source, i.e. a spot light or the sun. An extended source (e.g. a fluorescent lamp) will not reconstruct a hologram since it light is incident at each point at a wide range of angles, giving multiple reconstructions which will “wipe” one another out.

In this method, parallax in the vertical plane is sacrificed to allow a bright well-defined single colour re-constructed image to be obtained using white light. The rainbow holography recording process uses a horizontal slit to eliminate vertical parallax in the output image. The viewer is then effectively viewing the holographic image through a narrow horizontal slit. Horizontal parallax information is preserved but movement in the vertical direction produces colour rather than different vertical perspectives. Stereopsis and horizontal motion parallax, two relatively powerful cues to depth, are preserved.

The holograms found on credit cards are examples of rainbow holograms. These are technically transmission holograms mounted onto a reflective surface like a metalized polyethylene terephthalate substrate commonly known as PET.

Effects produced by lenticular printing, the pepper's Ghost illusion (or modern variants such as the Musion Eyeliner), and are another way to create the illusion of a 3D images on a planar surface. The Pepper's ghost technique, being the easiest to implement of these methods, is most prevalent in 3D displays that claim to be (or are referred to as) “holographic”. While the original illusion, used in theater, recurred to actual physical objects and persons, located offstage, modern variants replace the source object with a digital screen, which displays imagery generated with 3D computer graphics to provide the necessary depth cues. The reflection, which seems to float mid-air, is still flat, however, thus less realistic than if an actual 3D object was being reflected.

Lenticular printing is a technology in which a lenticular lens is used to produce images with an illusion of depth, or the ability to change or move as the image is viewed from different angles. Examples of lenticular printing include prizes given in Cracker Jack snack boxes that showed flip and animation effects such as winking eyes, and modern advertising graphics that change their message depending on the viewing angle. This technology was created in the 1940s but has evolved in recent years to show more motion and increased depth. Originally used mostly in novelty items and commonly called “flicker pictures” or “wiggle pictures,” lenticular prints are now being, used as a marketing tool to show products in motion. Recent advances in large-format presses have allowed for oversized lenses to be used in lithographic lenticular printing.

Photochromic lenses are lenses that darken on exposure to ultraviolet (UV) radiation. Once the UV is removed (for example by walking indoors), the lenses will gradually return to their clear state. Photochromic lenses may be made of glass, polycarbonate, or another plastic.

Photohromic lenses were developed by leading glass expert Roger Araujo at the Corning Glass Works Inc. in the 1960s, and created the first mass-produced variable tint lenses. The glass version of these lenses achieve their photochromic properties through the embedding of microcrystalline silver halides (usually silver chloride), or molecules in a glass substrate. Plastic photochromic lenses rely on organic photochromic molecules (for example oxazines and naphthopyrans) to achieve the reversible darkening effect. The reason these lenses darken in sunlight but not indoors under artificial light, is that room light does not contain the UV (short wavelength light) found in sunlight. Automobile windows also block UV so these lenses would darken less in a car. Lenses that darken in response to visible (rather than UV) light would avoid these issues, but they are not feasible for most applications. In order to respond to light, it is necessary to absorb it, thus the glass could not be made to be clear in its low-light state. This correctly implies photochromic lenses are not entirely transparent, specifically they filter out UV light. This does not represent a problem, because the human eye does not see in the UV spectrum.

With the photochromic material dispersed in the glass substrate, the degree of darkening depends on the thickness of glass, which poses problems with variable-thickness lenses in prescription glasses. With plastic lenses, the material is typically embedded into the surface layer of the plastic in a uniform thickness of up to 150 μm.

Typically, photochromic lenses darken substantially in response to UV light in less than one minute, and then continue to darken very slightly over the next fifteen minutes. The lenses fade back to clear along a similar pattern. The lenses will begin to clear as soon as they are away from UV light, and will be noticeably lighter within two minutes and mostly clear within five minutes. However, it normally takes more than fifteen minutes for the lenses to completely fade to their non-exposed state. A study by the Institute of Ophthalmology at the University College London has suggested that even in dark conditions photochromic lenses can absorb up to 20% of ambient light.

Because photochromic compounds fade back to their clear state by a thermal process, the higher the temperature, the less dark photochromic lenses will be. This thermal effect is called “temperature dependency” and prevents these devices from achieving true sunglass darkness in very hot weather. Conversely, photochromic lenses will get very dark in cold weather conditions, which makes them more suitable for snow skiers than beachgoers while outside. Once inside, away from the triggering UV light, the cold lenses take longer to regain their clear color than warm lenses.

A number of sunglass manufacturers/retailers (Intercast, Oakley, Serengeti Eyewear, Persol to name a few) offer products that use photochromism to make lenses that go from a dark to a darker state. Because these products are tinted in the bleached state, they are typically used only outdoors and are not considered general-purpose lenses.

See-through window graphics, technology that can be extended to this application are printed on mechanically perforated vinyl. This vinyl has a sticky adhesive on one side, protected by a peel away “release layer.” This perforated material, known as “window perf”, is made by many companies around the United States. All see-through window graphics are printed on basically the same material. The beauty of “window perf” is that it is incredibly low tech, comprised of 50% vinyl and 50% holes. Human eyes absorb light reflected from objects. When someone looks at the image on your window, their eyes absorb the light being reflected off of the printed image. The holes cannot be seen. The graphic looks like a solid image. The “sticky side” of the graphic which faces the glass, is black. Looking out from inside the vehicle, your eyes absorb light reflected off objects outside the vehicle, such as buildings, cars, trees, etc. Your eyes blend the black material on the inside with the images seen through the holes, creating the illusion that there is nothing on your window.

Glass and plastic can be coated to diminish the amount of ultraviolet radiation that passes through. Common uses of such coating include eyeglasses and automotive windows. Photographic filters remove ultraviolet to prevent exposure of the film or sensor by invisible light. UV curable coatings can be used to impart a variety of properties to polymeric surfaces, including glare reduction, wear or scratch resistance, anti-fogging, microbial resistance, chemical resistance. Computer screens, keyboards, cell phone surfaces, and most other personal electronic devices are treated with some type of UV-curable coating. Coatings are usually applied to plastic substrates via spray, dip, roll, flow and other processes. UV-curable coatings are often specified for plastic parts because the process does not require heat, which can distort the plastic shape.

Therefore embodiments of the present invention may be produced in the form of motor or bicycle helmet.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. The invention claimed is a three dimensional moving photo holographic image applied to sports equipment such as motorcycle helmets, windshield visors, snow goggles, essentially as described herein.

2. The invention of claim 1 wherein the equipment is a motorcycle helmet in which the visor portion thereof is imprinted with a holographic or lenticular image such that the image appears to be three dimensional and or moving from the viewer's perspective.

3. The invention of claim 2 in which the visor is photochromic.

4. The invention of claim 2 in which the visor is further treated to be UV light protectant or blocking.

5. The invention of claim 3 in which the visor is further treated to be UV light protectant or blocking

6. The invention of claim 1 wherein the image comprises a perforated vinyl sheet being the shape of a motorcycle helmet visor and imprinted with a holographic or lenticular image such that the image appears to be three dimensional and or moving from the viewer's perspective on the outside surface and further backed on the opposite or inside surface with adhesive for adherence to the visor such that the holographic or lenticular image is visible from the outside of the helmet.

7. The invention of claim 6 in which the visor is photo chromic.

8. The invention of claims 6 in which the visor is further treated to be UV light protectant or blocking.

9. The invention of claim 7 in which the visor is further treated to be UV light protectant or blocking

10. The invention claimed is motorcycle windshield that is imprinted with a holographic or lenticular image such that the image appears to be three dimensional and or moving from the viewer's perspective.

11. The invention of claim 10 wherein the image comprises a perforated vinyl sheet being the shape of a motorcycle windshield that is imprinted with a holographic or lenticular image such that the image appears to be three dimensional and or moving from the viewer's perspective on the outside surface and further backed on the opposite or inside surface with adhesive for adherence to the windshield such that the holographic or lenticular image is visible from the outside of the windshield.

12. The invention claimed is a pair of goggles that is imprinted with a holographic or lenticular image such that the image appears to be three dimensional and or moving from the viewer's perspective.

13. The invention of claim 12 in which the goggle lenses are photochromic.

14. The invention of claim 11, wherein the goggle lenses are further treated to be UV light protectant or blocking.

15. The invention of claim 12, wherein the goggle lenses are further treated to be UV light protectant or blocking.

16. The invention of claim 12 wherein the image comprises is a perforated vinyl sheet being the shape of goggle lenses that is imprinted with a holographic or lenticular image such that the image appears to be three dimensional and or moving from the viewer's perspective on the outside surface and further backed on the opposite or inside surface with adhesive for adherence to goggles such that the holographic or lenticular image is visible from the outside of the goggles.

17. The invention of claim 16 in which the goggle lenses are photochromic.

18. The invention of claim 16, wherein the goggle lenses are further treated to be UV light protectant or blocking.

19. The invention of claim 17, wherein the goggle lenses are further treated to be UV light protectant or blocking
